(19) United States
(12) Patent Application Publication
(10) Pub. No.: US 2025/0161451 A1
(43) Pub. Date: May 22, 2025

(54) ULTRASOUND-RESPONSIVE NANOSCALE DELIVERY PLATFORM FOR USE IN THE TREATMENT OF BIOFILM-ASSOCIATED DISEASES

(71) Applicants: Victor Choi, Richmond Hill (CA); Eleanor Phoebe Jane Stride, Oxford (GB); Dario Carugo, Reading (GB)

(72) Inventors: Victor Choi, Richmond Hill (CA); Eleanor Phoebe Jane Stride, Oxford (GB); Dario Carugo, Reading (GB)

(21) Appl. No.: 18/906,245

(22) Filed: Oct. 4, 2024

Related U.S. Application Data
(60) Provisional application No. 63/599,777, filed on Nov. 16, 2023.

(51) Int. Cl. A61K 41/00; A61K 9/51; A61K 31/55; A61K 31/555; A61K 31/65; A61K 31/7052; A61K 38/12; A61P 31/04

(52) U.S. Cl. CPC A61K 41/0028; A61K 9/5123; A61K 9/5146; A61K 31/55; A61K 31/555; A61K 31/65; A61K 31/7052; A61K 38/12; A61P 31/04

(57) ABSTRACT

An ultrasound-responsive nanoscale delivery platform for use in treating biofilm-associated diseases is provided. The inventors have found that the use of a phase-shift nanodroplet/antimicrobial complex platform confers a number of advantages when used in methods for antibiofilm therapy. Specifically, the inclusion of antimicrobials within the supramolecular structure of the ultrasound-responsive nanoscale delivery platform allowed for increased passive cellular and subcellular uptake, stimuli-responsive spatiotemporally controlled drug release, and significantly higher bacterial toxicity in both planktonic cultures and mature biofilms. This platform remains stable in storage at room temperature, in blood components, and does not significantly prematurely release its loaded cargo. The platform may be used as a novel treatment option in biofilm-associated diseases such as, but not limited to, bone and joint infections, cystic fibrosis-associated pleural infections, chronic wounds, and chronic urinary tract infections.

### CROSS-REFERENCE TO RELATED APPLICATIONS

The present invention is a U.S. Utility Patent Application that claims priority to U.S. Provisional Patent Application No. 63/599,777, filed on Nov. 16, 2023, the entire disclosure of which is incorporated herein by reference.

### TECHNICAL FIELD

The present invention relates to improvements in and relating to platforms of drug delivery and, in particular, to the treatment of diseases characterised by dynamic self-constructed microbial communities. More specifically, the invention relates to novel nanosized drug delivery vehicles carrying antimicrobials and to their use for biofilm-associated diseases.

### BACKGROUND

Most bacteria in chronic and nosocomial clinical infections are increasingly recognized to live within dynamic self-constructed microbial communities known as biofilms. These biofilms exhibit a physical diffusion barrier of an exopolysaccharide (EPS) matrix, non-metabolic dormant cells, and promote communication and interaction among bacteria (quorum sensing) to ensure their propagation and survival, making them markedly different from the planktonic bacteria used in traditional antimicrobial testing. As a result, many antimicrobials show minimal efficacy against biofilms at conventional dosages, exhibiting up to 1000× more resistance than planktonic-cased infections, rendering traditional antimicrobial therapy ineffective against chronic and localized infections.

Although there has been substantial growth in antibiofilm therapy research in recent years, there is a paucity of systemic therapies showing longitudinal biocidal effects in vivo, with no systemic therapy progressing beyond Phase I clinical trials. Particularly, many agents are unable to efficiently accumulate beyond the biofilm matrix or are retained poorly in it, leading to sub-therapeutic drug concentrations within the biofilm, reducing efficacy and promoting the development of AMR. To address this fundamental delivery constraint for a robust, efficacious, and universally applicable therapy, we have established an ultrasound-responsive nanoscale drug delivery platform capable of spatiotemporally controlled drug release and simultaneously promoting biofilm disruption whilst reducing toxic side-effects due to the shielding of the antimicrobial from off-target sites prior to reaching the desired infection site.

Ultrasound-activated drug delivery is an emerging non-invasive therapeutic platform with established safety and tolerability, building upon the fundamentals of diagnostic clinical ultrasound. Using therapeutic focused ultrasound (FUS), the delivery of relatively low frequency (1-5 MHz) bursts of sound waves can interact with exogenous gas-filled nuclei (microbubbles) to cause bubble acoustic cavitation (i.e. volumetric bubble oscillations). This focal bubble activity can in turn trigger the controlled local release of antimicrobials to allow for selective accumulation and extravasation to target bacterial communities. The large size, poor stability, and rapid destruction upon high-pressure ultrasound exposure of these microbubbles, however, greatly limits their clinical utility for systemic applications where permeability beyond the endothelial barrier is critical for reaching target tissue sites. To overcome this, efforts have been made to instead form phase change contrast agents (nanodroplets) by condensing the bubble gas core to the liquid state. This reduction in core density allows for a ˜5× reduction in particle size, increasing their stability in circulation, and their ability to extravasate and accumulate in target tissues. Upon vaporization by relatively high-pressure ultrasound, nanodroplets can covert back into echogenic gas-filled microbubbles for ultrasound-mediated drug transport and delivery. The acoustic energy associated with this phenomenon, termed acoustic droplet vaporization (ADV), can itself induce biofilm structure destruction and sloughing to remove cells from the protective EPS matrix, activating otherwise metabolically inactive “dormant” cells. Taken together, ultrasound-activated antimicrobial nanodroplet therapy holds potential to transiently disrupt biofilm communities whilst increasing delivery of bactericidal agents to target cells.

Despite these major advantages, previous studies exploring the co-delivery of vancomycin with a stimulated phase-shift nanodroplet platform showed a strong but incomplete bactericidal effect on MRSA biofilms in vitro. Although antimicrobial co-delivery shows promise, its efficacy has been limited by the dependence of free drug to bypass the biofilm barrier and reach the target site. Additionally, many promising antibiofilm agents in development, such as peptides and gene therapy, are limited by nuclease-mediated degradation and rapid clearance when introduced to the bloodstream. Inclusion of antimicrobials within the particle shell can therefore promote physiochemical and pharmacokinetic stability, improve local concentrations, and further increase antibiofilm activity in a particle-dependent manner.

### SUMMARY OF THE INVENTION

The inventors have established an ultrasound-responsive nanoscale delivery platform that leverages these advantages and exploits them for antibiofilm therapy. Specifically, the inventors have found that the use of this phase-shift nanodroplet/antimicrobial complex platform confers a number of advantages when used in methods for antibiofilm therapy. Specifically, it has been found that the inclusion of antimicrobials within the supramolecular structure of the ultrasound-responsive nanoscale delivery platform allowed for increased passive cellular and subcellular uptake, stimuli-responsive spatiotemporally controlled drug release, and significantly higher bacterial toxicity in both planktonic cultures and mature biofilms. Evidence is also provided to demonstrate that this platform remains stable in storage at room temperature, in blood components, and does not significantly prematurely release its loaded cargo.

The results provided herein illustrate the capacity of the ultrasound-responsive nanoscale delivery platform as a novel treatment option in biofilm-associated diseases such as, but not limited to, bone and joint infections, cystic fibrosis-associated pleural infections, chronic wounds, and chronic urinary tract infections. In other aspects, the biofilm-related disease is caused by a microbe such as a bacterium, a virus, a fungus, or a parasite. In further aspects, the biofilm-related disease comprises pneumonia, cystic fibrosis, otitis media, chronic obstructive pulmonary disease, a urinary tract infection, a periodontal disease, and/or a medical device-related infection.

Accordingly, in one embodiment, the present invention is directed to a phase-shift nanodroplet-antimicrobial agent platform effective for treating a biofilm-related disease in a subject in need thereof. In some aspects, the phase-shift nanodroplet is attached to or associated with at least one antimicrobial agent by covalent or non-covalent means. In other aspects, the phase-shift nanodroplet comprises a shell and a liquid core, and wherein the shell comprises a material capable of forming and maintaining a layer at an interface between the liquid core and an external medium. In other aspects, the material is a surfactant or a polymer. In further aspects, the platform has a diameter in the range of 1 nm to 10 μm.

In another embodiment, the present invention is directed to a pharmaceutical composition comprising the antimicrobial-loaded phase-shift nanodroplet platform as previously described, and at least one pharmaceutical carrier or excipient, wherein the pharmaceutical composition is formulated for parenteral, oral, intranasal, or topical administration.

In another embodiment, the present invention is directed to a method for treating a biofilm-related disease in a subject in need thereof, comprising:
a) administering an effective amount of the pharmaceutical composition of claim 5 to a treatment area in or on the subject; and
b) subjecting the treatment area to ultrasound irradiation.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1A shows that, when condensed, azithromycin-loaded phase-shift nanodroplets had an average diameter of 100-300 nm, and concentration of approximately 10¹¹ particles/mL:

FIG. 1B shows that azithromycin-loaded precursor microbubbles had an average diameter of 300-800 nm, average volume of 2-6 μm³, and concentration of approximately 10¹¹ particles/mL;

FIG. 1C shows that prepared pharmaceutical compositions of azithromycin-loaded phase-shift nanodroplets were observed to be stable for 120 days when stored at room temperature;

FIG. 2A shows that, when condensed, besifloxacin-loaded phase-shift nanodroplets had an average diameter of 100-300 nm, and concentration of approximately 10¹¹ particles/mL;

FIG. 2B shows that besifloxacin-loaded precursor microbubbles had an average diameter of 400-1500 μm, average volume of 2-8 μm³, and concentration of approximately 10¹¹ particles/mL;

FIG. 2C shows that, when evaluating the stability of besifloxacin-loaded phase-shift nanodroplets in serum, up to 80% of the initial concentration was retained after 100 hours incubation, with less than 1% of the population being above 0.8 μm, representing spontaneous vaporization;

FIG. 3A shows that, when condensed, polymyxin B-loaded phase-shift nanodroplets had an average diameter of 100-300 nm, and concentration of approximately 10¹¹ particles/mL;

FIG. 3B shows that polymyxin B-loaded precursor microbubbles had an average diameter of 400-800 μm, average volume of 2-6 μm³, and concentration of approximately 10¹¹ particles/mL;

FIG. 4A shows that, when condensed, ruthenium polypyridyl complex-loaded phase-shift nanodroplets had an average diameter of 200-400 nm, and concentration of approximately 10¹¹ particles/mL;

FIG. 4B shows that ruthenium polypyridyl complex-loaded precursor microbubbles had an average diameter of 500-900 μm, average volume of 2-7 μm³, and concentration of approximately 10¹¹ particles/mL;

FIGS. 5A-5B show a statistically significant reduction in bacterial planktonic viability across all tested clinical isolates (symbols) for S. aureus (FIG. 5A) and E. coli (FIG. 5B) treated with combined antimicrobial-loaded phase-shift nanodroplets and focused ultrasound (light grey), when compared with either antimicrobial alone (black), or antimicrobial-loaded nanodroplets alone (dark grey). Plots depict the antimicrobial concentration required by each treatment to achieve the Minimum Inhibitory Concentration (MIC) per European Committee on Antimicrobial Susceptibility Testing (EUCAST) standards and metabolic viability (resazurin), and Minimum Bactericidal Concentration (MBC: defined as a 3-log reduction in culturable units per millilitre). Each data point corresponds to a different bacterial clinical isolate, with each point representing three biological replicates. Bars indicate mean values across each tested isolate;

FIGS. 6A-6B show a statistically significant increase in overall antimicrobial cellular uptake when planktonic bacteria were incubated with antimicrobial-loaded phase-shift nanodroplets when compared to antimicrobial alone in E. coli (FIG. 6A) and S. aureus (FIG. 6B). Each data point corresponds to a different bacterial clinical isolate, with each point representing three biological replicates. Bars indicate mean values across each tested isolate;

FIGS. 7A-7B show a statistically significant reduction in biofilm viability across all tested clinical isolates for S. aureus (FIG. 7A) and E. coli (FIG. 7B) treated with combined antimicrobial-loaded phase-shift nanodroplets and focused ultrasound (light grey), when compared with antimicrobial alone (black), or antimicrobial-loaded nanodroplets alone (dark grey). Plots depict the antimicrobial concentration required by each treatment to achieve the Minimum Biofilm Eradication Concentration (MBEC) when measured through biomass reduction (90% reduction in safranin), metabolic viability (80% reduction in resazurin), or culturability (3-log reduction in culturable units per millilitre). Persister elimination is defined as the antimicrobial concentration required for complete eradication below the detection limit of 1 culturable unit per millilitre. Each data point corresponds to a different bacterial clinical isolate, with each point representing three biological replicates. Bars indicate mean values across each tested isolate;

FIG. 7C shows a statistically significant reduction in cell viability and persister cell percentage in both the outflow fraction during treatment (first column) and residual cell (second columns) fractions of S. aureus, E. coli, and P. aeruginosa biofilms grown in simulated infection conditions representative of joint infection, urinary tract infection, and chronic wound/cystic fibrosis, respectively, when treated with antimicrobial-loaded phase-shift droplets (ND/FUS) compared to antimicrobial alone or ultrasound alone (FUS).

FIGS. 8A-8B show a significantly higher accumulation within the cytoplasmic compartment of biofilm-residing bacteria when comparing nanodroplet-encapsulated antimicrobials to free drug formulations in E. coli (FIG. 8A) and S. aureus (FIG. 8B). Each data point corresponds to a different bacterial clinical isolate, with each point representing three biological replicates. Bars indicate mean values across each tested isolate

FIG. 9A shows that, when condensed, minocycline-loaded phase-shift nanodroplets had an average diameter of 100-300 nm, and concentration of approximately 10¹² particles/mL; and

FIG. 9B shows that Franz diffusion cell testing of the nanodroplets of FIG. 9A through a model biofilm suggests passive release of nanodroplets from the gel through the biofilm within 2 hours of incubation.

### DETAILED DESCRIPTION OF THE INVENTION

The subject matter of the present invention now will be described more fully hereinafter, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions. Therefore, it is to be understood that the subject matter of the present invention is not to be limited to the specific embodiments disclosed and that modifications and other embodiments are intended to be included within the scope of the appended claims.

### Ultrasound-Responsive Nanoscale Delivery Platform for Use in the Treatment of Biofilm-Associated Diseases

In the broadest sense, the invention provides an ultrasound-responsive nanoscale delivery platform for use in the treatment of biofilm-associated diseases.

In one embodiment, the present invention is directed to a phase-shift nanodroplet-antimicrobial agent platform effective for treating a biofilm-related disease in a subject in need thereof. In some aspects, the phase-shift nanodroplet is attached to or associated with at least one antimicrobial agent by covalent or non-covalent means. In other aspects, the phase-shift nanodroplet comprises a shell and a liquid core, and wherein the shell comprises a material capable of forming and maintaining a layer at an interface between the liquid core and an external medium. In other aspects, the material is a surfactant or a polymer. In further aspects, the platform has a diameter in the range of 1 nm to 10 μm.

In another embodiment, the present invention is directed to a pharmaceutical composition comprising the antimicrobial-loaded phase-shift nanodroplet platform as previously described, and at least one pharmaceutical carrier or excipient, wherein the pharmaceutical composition is formulated for parenteral, oral, intranasal, intraarticular, or topical administration.

In another embodiment, the present invention is directed to a method for treating a biofilm-related disease in a subject in need thereof, comprising:
a) administering an effective amount of the pharmaceutical composition of claim 5 to a treatment area in or on the subject; and
b) subjecting the treatment area to ultrasound irradiation.

As used herein, the term “biofilm” is intended to refer to a dynamic self-constructed accumulation of microorganisms that produce a matrix of extracellular biopolymers (also referred to herein as “extracellular polysaccharides” (EPS)). The ultrasound-responsive nanoscale delivery platform comprises a nano-sized phase-shift nanodroplet attached to, loaded with, or otherwise associated with at least one antimicrobial agent. Although ultrasound is unnecessary to achieve enhanced cellular uptake and biological response, it comprises ultrasound-responsive properties to allow for synergistic biofilm dispersal. Specifically, it is possible that the liquid core within the nanodroplet platform can be vaporized by ultrasound to form gaseous core ultrasound-contrast agents (microbubbles), which can in-turn be undergo dynamic behaviour and/or rupture to enhance drug delivery and drug release at the desired target site.

As used herein, the term “antimicrobial” is intended to broadly encompass any biological, chemical, or small molecule compound utilised, either clinically or pre-clinically, that either kills microorganisms (e.g. bacteria), or stops their growth. For use in this invention, suitable classes of antimicrobials and examples within those classes include antibiotics, antivirals, antifungals, anti-parasitic agents, and microorganisms.

Accordingly, in some embodiments, an antimicrobial agent is an antibiotic. The terms “antibiotic,” “antibiotic agent,” and “antibacterial agent” may be used interchangeably.

In some embodiments, the antibiotic is a macrolide, an aminoglycoside, a tetracycline, a peptide, a glycopeptide, a penicillin, a cephalosporin, a quinolone, a fluoroquinolone or a rifampin. An antibiotic may also be a pharmaceutically acceptable salt of any molecule described above, or a combination of these molecules.

In some embodiments, the antibiotic is an aminoglycoside. In some embodiments, the antibiotic is apramycin, gentamicin, kanamycin, neomycin, paromomycin, plazomicin, spectinomycin, a combination thereof, or a pharmaceutically acceptable salt thereof.

In some embodiments the antibiotic is a macrolide. In some embodiments, the antibiotic is dirithromycin, erythromycin, a combination thereof, or a pharmaceutically acceptable salt thereof.

In some embodiments the antibiotic is a tetracycline or a pharmaceutically acceptable salt thereof. In some embodiments the antibiotic is doxycycline, tetracycline, a combination thereof, or a pharmaceutically acceptable salt thereof.

In some embodiments the antibiotic is a penicillin or a pharmaceutically acceptable salt thereof. In some embodiments, the antibiotic is ampicillin, amoxicillin, cloxacillin, piperacillin, oxacillin, a combination thereof, or a pharmaceutically acceptable salt thereof.

In some embodiments the antibiotic is a quinolone, fluoroquinolone, or a pharmaceutically acceptable salt thereof. In some embodiments, the antibiotic is ciprofloxacin, besifloxacin, enoxacin, nalidixic acid, norfloxacin, levofloxacin, moxifloxacin, pefloxin, a combination thereof, or a pharmaceutically acceptable salt thereof.

In some embodiments the antibiotic is a cephalosporin or a pharmaceutically acceptable salt thereof. In some embodiments the antibiotic is ceftriaxone, cefoperazone, a combination thereof, or a pharmaceutically acceptable salt thereof.

In some embodiments the antibiotic is a peptide, glycopeptide, or a pharmaceutically acceptable salt thereof. In some embodiments, the antibiotic is vancomycin, polymyxin B, a combination thereof, or a pharmaceutically acceptable salt thereof. In some embodiments, the antibiotic is a cathelicidin peptide.

In some embodiments, the antibiotic is chloramphenicol, dirithromycin, erythromycin, doxycycline, tetracycline, linezolid, bacitracin, fosfomycin, fosmidomycin, ampicillin, amoxicillin, cloxacillin, piperacillin, oxacillin, ceftriaxone, cefoperazone, vancomycin, polymyxin B, ciprofloxacin, besifloxacin, enoxacin, nalidixic acid, norfloxacin, levofloxacin, moxifloxacin, pefloxin, novobiocin, pentamidine, rifampicin, trimethoprim, sulfamethoxazole, a combination thereof, or a pharmaceutically acceptable salt thereof.

In some embodiments, the antibiotic is apramycin, neomycin, paromycin, spectinomycin, chloramphenicol, dirithromycin, erythromycin, doxycycline, tetracycline, linezolid, bacitracin, fosfomycin, fosmidomycin, ampicillin, amoxicillin, cloxacillin, a combination thereof, or a pharmaceutically acceptable salt thereof.

In some embodiments, the antibiotic is erythromycin, dirithromycin, levofloxacin, norfloxacin, ciprofloxacin, enoxacin, moxifloxacin, besifloxacin polymyxin B, a combination thereof, or a pharmaceutically acceptable salt thereof.

In some embodiments, the antibiotic is cefoperazone, novobiocin, ampicillin, cloxacillin, oxacillin, doxycycline or a pharmaceutically acceptable salt thereof.

In some embodiments, an antibiotic agent is Amikacin, Apramycin, Gentamicin, Kanamycin, Neomycin, Tobramycin, Paromomycin, Streptomycin, Spectinomycin, Ertapenem, Doripenem, Imipenem/Cilastatin, Meropenem, Cefadroxil, Cefazolin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Teicoplanin, Vancomycin, Telavancin, Clindamycin, Lincomycin, Lipopeptide, Daptomycin, Azithromycin, Clarithromycin, Dirithromycin, Erythromycin, Roxithromycin, Troleandomycin, Telithromycin, Spiramycin, Aztreonam, Linezolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Bacitracin, Colistin, Polymyxin B, Besifloxacin, Ciprofloxacin, Enoxacin, Gatifloxacin, Gemifloxacin, Levofloxacin, Lomefloxacin, Moxifloxacin, Nalidixic acid, Norfloxacin, Ofloxacin, Pefloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Sulfonamidochrysoidine, Demeclocycline, Doxycycline, Minocycline, Oxytetracycline, Tetracycline, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim, Clofazimine, Dapsone, Capreomycin, Cycloserine, Ethambutol, Ethionamide, Isoniazid, Pyrazinamide, Rifampicin, Rifabutin, Rifapentine or Streptomycin, or a pharmaceutically acceptable salt thereof.

In other embodiments, the antibiotic kills or slows the growth of bacteria that cause diseases that include but are not limited to diphtheria (e.g., Corynebacterium diphtheria), pertussis (e.g., Bordetella pertussis), anthrax (e.g., Bacillus anthracia), typhoid, plague, shigellosis (e.g., Shigella dysenteriae), botulism (e.g., Clostridium botulinum), tetanus (e.g., Clostridium tetani), tuberculosis (e.g., Mycobacterium tuberculosis), bacterial pneumonias (e.g., Haemophilus influenzae), cholera (e.g., Vibrio cholerae), salmonellosis (e.g., Salmonella typhi), peptic ulcers (e.g., Helicobacter pylori), Legionnaire's Disease (e.g. Legionella spp.), and Lyme disease (e.g. Borrelia burgdorferi). Other pathogenic bacteria include Escherichia coli, Clostridium perfringens, Clostridium difficile, Pseudomonas aeruginosa, Staphylococcus aureus, and Streptococcus pyogenes. Further examples of bacteria include Staphylococcus epidermidis, Staphylococcus sp., Streptococcus pneumoniae, Streptococcus agalactiae, Enterococcus sp., Bacillus cereus, Bifidobacterium bifidum, Lactobacillus sp., Listeria monocytogenes, Nocardia sp., Rhodococcus equi, Erysipelothrix rhusiopathiae, Propionibacterium acnes, Actinomyces sp., Mobiluncus sp., Peptostreptococcus sp., Neisseria gonorrhoeae, Neisseria meningitides, Moraxella catarrhalis, Veillonella sp., Actinobacillus actinomycetemcomitans, Acinetobacter baumannii, Brucella sp., Campylobacter sp., Capnocytophaga sp., Cardiobacterium hominis, Eikenella corrodens, Francisella tularensis, Haemophilus ducreyi, Helicobacter pylori, Kingella kingae, Legionella pneumophila, Pasteurella multocida, Klebsiella granulomatis, Enterobacteriaceae, Citrobacter sp., Enterobacter sp., Klebsiella pneumoniae, Proteus sp., Salmonella enteriditis, Shigella sp., Serratia marcescens, Yersinia enterocolitica, Yersinia pestis, Aeromonas sp., Plesiomonas shigelloides, Vibrio cholerae, Vibrio parahaemolyticus, Vibrio vulnificus, Acinetobacter sp., Flavobacterium sp., Burkholderia cepacia, Burkholderia pseudomallei, Xanthomonas maltophilia, Stenotrophomonas maltophila, Bacteroides Bacteroides sp., Prevotella sp., Fusobacterium sp., and Spirillum minus. Antibiotics are agents used to kill, inhibit, or slow the growth of bacteria or other microorganisms and include, but are not limited to, aminoglycosides (such as amikacin, gentamicin, kanamycin, neomycin, netilmicin, tobramycin, paromomycin, streptomycin, and spectinomycin), ansamycins (such as geldanamycin, herbimycin, and rifaximin), carbacephem (such as loracarbef) carbapenems (such as ertapenem, doripenem, imipenem/cilastatin, and meropenem), cephalosporins (such as cefadroxil, cefaxolin, cefalotin (cefalothin), cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil and ceftobiprole), glycopeptides (such as teicoplanin, vancomycin, telavancin, dalbavancin, and oritavancin), lincosamides (such as clindamycin and lincomycin), lipopetides (such as daptomycin), macrolides (such as azithromycin, clarithromycin, dirithromycin, erythromycin, roxithromycin, troleandomycin, telithromycin, and spiramycin), monobactams (such as aztreonam), nitrofurans (such as furazolidone, and nitrofurantoin), oxazolidinones (such as linezolid, posizolid, radezolid, and torezolid), penicillins (such as amoxicillin, ampicillin, azlocillin, carbenicillin, cloxacillin, dicloxacillin, flucloxacillin, mexlocillin, methicillin, nafcillin, oxacillin, penicillin G, penicillin V, piperacillin, temocillin, and ticarcillin), polypeptides (such as bacitracin, colistin, and polymyxin B), quinolones/fluoroquinolones (such as ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, and temafloxacin), sulfonamides (such as mafenide, sulfacetamide, sulfadiazine, silver sulfadiazine, sulfadimethoxine, sulfamethizole, sulfamethoxazole, sulfanilamide, sulfasalazine, sulfisoxazole, trimethoprim-sulfamethoxazole, and sulfonamidochrysoidine), tetracylines (such as demeclocycline, doxycycline, minocycline, oxytetracycline, and tetracycline), antimycobacteria (such as clofazimine, dapsone, capreomycin, cycloserine, ethambutol, ethionamide, isoniazid, pyrazinamide, rifampicin, rifabutin, rifapentine, streptomycin), arsphenamine, chloramphenicol, fosfomycin, fusidic acid, metronidazole, mupirocin, platensimycin, quinupristin/dalfopristin, thiamphenicol, tigecycline, tinidazole, trimethoprim, and teixobactin.

In other embodiments, the antimicrobial agent is an antiviral agent.

In some embodiments, an antiviral agent is Abacavir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Norvir, Oseltamivir, Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir or Zidovudine, or a pharmaceutically acceptable salt thereof.

In other embodiments, the antiviral agent kills or slows the growth of viruses that include, but are not limited to, measles, mumps, rubella, poliomyelitis, hepatitis (e.g. hepatitis A, B, C, delta, and E viruses), influenza, adenovirus, rabies, yellow fever, Epstein-Barr virus, herpesviruses, papillomavirus, Ebola virus, influenza virus, Japanese encephalitis, dengue virus, hantavirus, Sendai virus, respiratory syncytial virus, othromyxoviruses, vesicular stomatitis virus, visna virus, cytomegalovirus, and human immunodeficiency virus (HIV). Antivirals are agents used to kill, inhibit, or slow the growth of viruses and include, but are not limited to, anti-(HIV) agents (such as abacavir, didanosine, emtricitabine, lamivudine, stavudine, tenofovir disoproxil fumarate, zidovudine, delavirdine, efavirenz, etravirine, nevirapine, rilpivirine, atazanavir, darunavir, fosamprenavir, indinavir, nelfinavir, ritonavir, saquinavir, tipranavir, enfuvirtide, maraviroc, dolutegravir, elvitegravir, raltegravir, cobicistat, and combinations thereof), anti-influenza virus agents (such as zanamivir, oseltamivir phosphate, peramivir, amantadine, and rimantadine), anti-herpes virus agents (such as acyclovir, valacyclovir, penciclovir, idoxuridine, vidarabine, trifluridine, foscarnet and famciclovir), anti-hepatitis virus agents (such as adefovir, lamivudine, telbivudine, tenofovir, famciclovir, entecavir, ribavirin, telaprevir, simeprevir, sofosbuvir, ledipasvir, ombitasvir, paritaprevir, ritonavir, dasabuvir, and boceprevir), anti-cytomegalovirus (CMV) agents (such as ganciclovir, cidofovir, valganciclovir, foscarnet, maribavir, and leflunomide), anti-respiratory syncytial virus (RSV) agents (such as ribavirin and palivizumab), and anti-varicella-zoster virus (VSV) agents (such as acyclovir, valacyclovir, penciclovir, famciclovir, brivudin, foscarnet, and vidarabine).

In other embodiments, the antimicrobial agent is an anti-fungal agent.

In some embodiments, an anti-fungal agent is Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Posaconazole, Propiconazole, Ravuconazole, Terconazole, Voriconazole, Abafungin, Amorolfin, Butenafine, Naftifine, and Terbinafine, Anidulafungin, Caspofungin, Micafungin, Aurones, Benzoic acid, Ciclopirox, Flucytosine, Griseofulvin, Haloprogin, Tolnaftate, Undecylenic acid, Crystal violet, Orotomide or Miltefosine, or a pharmaceutically acceptable salt thereof.

In other embodiments, the anti-fungal agents kill or slow the growth of fungi that include but are not limited to Acremoniuin spp., Aspergillus spp., Epidermophytoni spp., Exophiala jeanselmei, Exserohilunm spp., Fonsecaea compacta, Fonsecaea pedrosoi, Fusarium oxysporum, Basidiobolus spp., Bipolaris spp., Blastomyces derinatidis, Candida spp., Cladophialophora carrionii, Coccoidiodes immitis, Conidiobolus spp., Cryptococcus spp., Curvularia spp., Fusarium solani, Geotrichum candidum, Histoplasma capsulatum var. capsulatum, Histoplasma capsulatum var. duboisii, Hortaea werneckii, Lacazia loboi, Lasiodiplodia theobromas, Leptosphaeria senegalenisis, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomycete fungi, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Absidia coryinbifera, Rhizomucor pusillus, and Rhizopus arrhizus. Antifungals are agents used to kill, inhibit, or slow the growth of fungi and include, but are not limited to, polyene antifungals (such as amphotericin B, candicin, filipin, hamycin, natamycin, nystatin, and rimocidin), azole antifungals (such as abafungin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, and terconazole, voriconazole), and echinocandins (such as anidulafungin, caspofungin, and micofungin).

In some embodiments, the antimicrobial agent is an anti-parasitic agent.

In some embodiments, an anti-parasitic agent is Nitazoxanide, Melarsoprol, Eflornithine, Metronidazole, Tinidazole, Miltefosine, Ancylostoma caninum, Mebendazole, Pyrantel pamoate, Thiabendazole, Diethylcarbamazine, Ivermectin, Niclosamide, Praziquantel, Albendazole, Antitrematodes, Praziquantel, Rifampin, Amphotericin B or Fumagillin, or a pharmaceutically acceptable salt thereof. Parasites include, but are not limited to, protozoa, nematodes, cestodes, trematodes, and other parasites, such as those responsible for diseases, including, but not limited to, malaria (e.g. Plasmodium falciparum), hookworm, tapeworms, helminths, whipworms, ringworms, roundworms, pinworms, ascarids, filarids, onchocerciasis (e.g., Onchocerca volvulus), schistosomiasis (e.g. Schistosoma spp.), toxoplasmosis (e.g. Toxoplasma spp.), trypanosomiasis (e.g. Trypanosoma spp.), leishmaniasis (Leishmania spp.), giardiasis (e.g. Giardia lamblia), amoebiasis (e.g. Entamoeba histolytica), filariasis (e.g. Brugia malayi), and trichinosis (e.g. Trichinella spiralis). Antiparasitics are agents used to kill, inhibit, or slow the growth of parasites and include, but are not limited to, antinematodes (such as mebendazole, pyrantel pamoate, thiabendazole, diethylcarbamazine, and ivermectin), anticestodes (such as niclosamide, praziquantel, and albendazole), antitrematodes (such as praziquantel), antiamoebics (such as rifampin and amphotericin B), and antiprotozoals (such as melarsoprol, eflornithine, metronidazole, tinidazole, and miltefosine).

Pharmaceutically acceptable salts, derivatives, or analogues of any of these compounds may also be used. The specific choice of antimicrobial in each situation is dependent on various clinical factors, including the nature of the infection, the patient to be treated, and the resistance profile of the bacterium etc, but can be readily selected by those skilled in the art.

The antimicrobial agent(s) may be linked to the phase-shift nanodroplet through covalent or non-covalent means, e.g. electrostatic interaction, van der Waals forces, and/or hydrogen bonding. Examples of methods which may be used to covalently link a phase-shift nanodroplet to antimicrobial agent(s) include but are not limited to: (a) carbodiimide based coupling methods, whereupon the shell contains either an amine or carboxylic acid functionality and is covalently linked to an antimicrobial containing either a carboxylic acid or amine functionality, resulting in the formation of ester or amide bonds. (b) “CLICK” reactions whereupon azide or acetylene functionalized phase-shift nanodroplet shells are reacted with an antimicrobial having either an acetylene or azide functionality. (c) Schiff base formation whereupon aldehyde or amine functionalized phase-shift nanodroplet shells are reacted with an antimicrobial having either an amine or aldehyde functionality. (d) Biotin-avidin linkages whereupon biotinylated or avidin-functionalized phase-shift nanodroplet shells are reacted with an antimicrobial having either a biotin or avidin functionality. (e) Michael addition reaction whereupon a Michael donor or α,β-unsatured carbonyl functionalized phase-shift nanodroplet shell is reacted with either a Michael donor or Michael acceptor-functionalized antimicrobial to form a carbon-carbon bond Michael adduct. In one embodiment, a cationic antimicrobial is electrostatically bound to the anionic outer shell of the nanodroplet. In another embodiment, a hydrophobic antimicrobial is bound within the lipid layer of the nanodroplet. In another embodiment, an antimicrobial is non-covalently linked to a phospholipid through strain promoted alkyne-azide cycloaddition (SPAAC) biorthogonal reactions, making up the nanodroplet shell.

Antimicrobials may also be coupled to the phase-shift nanodroplet alternatively through the lipid directly, using any of the methods described above, such that the antimicrobial is directly incorporated within the lipid shell during the production of the phase-shift nanodroplet.

As used herein, the term “phase-shift nanodroplet” is intended to refer to a nanosphere comprising a shell having an approximately spherical shape and which surrounds an internal void comprising a volatile liquid. The shape of the nanodroplet is, however, not essential to the invention and as such should not be considered limiting. The “shell” refers to the membrane which surrounds the internal void of the phase-shift nanodroplet. The size of the nanodroplet should be of such a size to permit its passage through either the pulmonary system, urinary tract, or pleural space following administration. Phase-shift nanodroplets typically have a diameter on production of less than 10 μm although they are preferably below 1 μm for increased extravasation, and more preferably below 200 nm to bypass the EPS matrix. The shell of the nanodroplet will vary in thickness depending on material and is not essential provided that the shell can perform the desired function of retaining the liquid core.

Materials used to form phase-shift nanodroplets are typically biocompatible and suitable materials are well known for those expert in the art. Typically, the shell of the microbubble will comprise either a surfactant, polymer, or any other material capable of forming and maintaining a layer at the interface between the liquid core and the external medium of which it is housed (e.g. blood or aqueous solution). Any combination of materials may be used as suitable. Lipids are preferentially used for this application, including lysolipids; phosphatidylcholine; distearoylphosphatidylcholine, dipalmitoylphosphatidylcholine, dimyristoylphosphatidylcholine, dibehenoylphosphatidylcholine, diarachidoylphosphatidylcholine, egg phosphatidylcholine, or any mixed acyl chain variations of the previous; phosphatidylethanolamine; distearoylphosphatidylethanolamine, dipalmitoylphosphatidylethanolamine, dimyristoylphosphatidylethanolamine, dibehenoylphosphatidylethanolamine, diarachidoylphosphatidylethanolamine, egg phosphatidylethanolamine, or any mixed acyl chain variations of the previous and/or any conjugates to polyethylene glycol 2000 or 5000 to the previous; phosphatidylserine; distearoylphosphatidylserine, dipalmitoylphosphatidylserine, dimyristoylphosphatidylserine, brain phosphatidylserine, or any mixed acyl chain variations of the previous; phosphatidylglycerol, lysyl phosphatidylglycerol, egg phosphatidylglycerol; phosphatidic acid, distearoyl phosphatidic acid, dipalmitoyl phosphatidic acid, dimyristoyl phosphatidic acid, dibehenoyl phosphatidic acid, diarachidoyl phosphatidic acid, egg phosphatidic acid, or any mixed acyl chain variations of the previous; phosphoinositides; phosphatidylserines; cardiolipins; cholesteryl spermine; 2,3-dioleyloxy-N-[2-(sperminecarboxamido)ethyl]-N,N-dimethyl-1-propanaminium HCl salt; N1-[2-((1S)-1-[(3-aminopropyl)amino]-4-[di(3-amino-propyl)amino]butylcarboxamido)ethyl]-3,4-di[oleyloxy]-benzamide; palmitic acid; stearic acid; arachidonic acid; oleic acid; lipids bearing polymers such as polyethyleneglycol, chitin, hyaluronic acid; lipids bearing sulfonated mono-, di-, oligo- or polysaccharides; cholesterol, cholesterol sulfate; tocopherol hemisuccinate, lipids with ether and ester-linked fatty acids, polymerized lipids, and ethylenediaminetetraacetic acid-lipid conjugates. Suitable lipids may be selected based on their ability to enhance stability of the nanodroplet platform in serum and prevent the spontaneous vaporization of its volatile liquid core.

Polymer materials which are suitable for use in forming the shell include proteins, in particular albumin, more particularly human serum albumin, bovine serum albumin, ovalbumin, mouse serum albumin, rat serum albumin, chicken serum albumin, porcine serum albumin, or combinations thereof. Other biocompatible polymers which may be used include poly(vinyl alcohol) (PVA), poly(D,L-lactide-co-glycolide) (PLGA), polyglycolic acid (PGA), polylactic acid (PLA), polycaprolactone (PCL), poly(N-isopropylacrylamide) (PNIPAAm), cyanoacrylate, poloxamers (Pluronics®), or combinations thereof.

Any of the nanodroplet shell formulations described herein may comprise further components which aid in the delivery of the phase-shift nanodroplet to the target site. For example, these may be functionalized to enhance delivery beyond the EPS matrix or by incorporating targeting ligands able to bind to target sites. Examples of suitable targeting agents include antibodies and antibody fragments, cell adhesion molecules and their receptors, aptamers, cytokines, growth factors, and receptor ligands. Such agents may be incorporated onto the nanodroplet shell using methods known in the art such as covalent coupling or avidin-biotin complexation.

As used herein, the term “liquid core” encompasses substances which are in liquid form at ambient temperature and pressure, whether naturally occurring or in a superheated state whereby the substance occurs naturally in gaseous state but is otherwise condensed into the liquid state for preparation. Substances suitable for incorporation within the core of the phase-shift nanodroplet include perfluorocarbons (e.g. perfluoroalkanes such as perfluoromethane, perfluoroethane, perfluoropropanes, perfluorobutanes, perfluoropentanes, perfluorohexanes and perfluoroheptanes; perfluoroalkenes such as perfluoropropene, perfluorobutenes; and perfluorocycloalkanes such as perfluorocyclobutane) and inert gases (e.g. xenon, krypton). Gases may also be dissolved within the liquid core for enhanced biological effect. Examples suitable for application include oxygen, nitric oxide, monoxide, carbon dioxide, and volatile anaesthetics such as nitrous oxide, halothane, isoflurane, or sevoflurane.

Methods for the formation of phase-shift nanodroplets may be prepared by those known in the art. Such methods include the formation of a suspension of a superheated liquid encompassed in the presence of the selected shell material. Techniques used to form this may include the condensation of precursor gas-filled microbubbles, sonication, extrusion and microfluidic processing. In the case of condensation, an aqueous suspension of formed precursor microbubbles is prepared using identical materials as the phase-shift nanodroplet. The condensation transition may be induced by physical and/or chemical means through a reduction in temperature and/or increase in pressure.

The antimicrobial-loaded phase-shift nanodroplet platform described herein may be used in treatment of biofilm-related diseases. As used herein, the term “biofilm-related disease” refers to a disease, disorder, and/or condition caused by a microbe (e.g., a bacterium, a virus, a fungus, or a parasite) that includes a tissue-related infection or a medical device-related infection. In some aspects, medical device-related infections include those grown on ventricular derivations; contact lenses; endotracheal tubes; central venous catheters; prosthetic cardiac valves, pacemakers, and vascular grafts; tissue fillers; breast implants; peripheral vascular catheters; urinary catheters; orthopaedic implants and prosthetic joints. In some aspects, tissue-related infections include acne, chronic otitis media, chronic sinusitis, chronic tonsillitis, periodontal disease (e.g., dental plaque, dental caries, and gingivitis), chronic laryngitis, endocarditis, lung infections (e.g., pneumonia), chronic obstructive pulmonary disease, cystic fibrosis, kidney stones, biliary tract infections, urinary tract infections, osteomyelitis, and chronic wounds. See also, e.g., Vestby L K, Grønseth T, Simm R, & Nesse LL. Bacterial Biofilm and its Role in the Pathogenesis of Disease. Antibiotics (Basel), 9(2):59 (2020); Gondil, V. S., & Subhadra, B. Biofilms and Their Role on Diseases. BMC Microbiol., 23:203 (2023); Del Pozo JL. Biofilm-related disease. Expert Rev. Anti Infect. Ther. 16(1):51-65 (2018); Maciá MD, Del Pozo JL, Díez-Aguilar M, & Guinea J. Microbiological diagnosis of biofilm-related infections. Enferm. Infecc. Microbiol. Clin. (Engl. Ed). 36(6):375-381 (2018); Alessandra, O, Stefani, S, Venditti, M, & Di Domenico, EG. Biofilm-Related Infections in Gram-Positive Bacteria and the Potential Role of the Long-Acting Agent Dalbavancin. Frontiers in Microbiology. 12: 749685 (2021)

For use in treatment of biofilm-related disease, the phase-shift nanodroplet complexes will be provided in a pharmaceutical composition together with at least one pharmaceutically acceptable carrier or excipient. The composition for use may be formulated using techniques well known to those in the art.

The route of administration will depend on the intended use and indication of treatment. These may be administered either systemically or topically and may thus be provided either in a form adapted for parenteral administration (e.g. by intradermal, subcutaneous, intraperitoneal, or intravenous injection), oral administration, intranasal administration, or topical administration. In case of each, suitable suspensions, solutions, creams, ointments, emulsions, sprays, or oral dosage forms containing the active phase-shift nanodroplet complex may be administered, together with one or more inert carriers or excipients. Suitable carriers or excipients may include, but are not limited to, diluents (e.g. saline, sterile water, phosphate buffered saline, sugar compounds), lubricants (talc, mineral oil, silicon dioxide, steric acid), wetting agent (polysorbates, sodium lauryl sulfate), binder agents (natural and synthetic polymers, starches, sugars), disintegrants (starch, cellulose derivatives), emulsifiers (sodium lauryl sulfate, parrafin, glycol, glyercols), preservatives (benzyl alcohol, parabens). The compositions may be sterilized by conventional sterilisation techniques.

In another aspect of the invention, the pharmaceutical composition of the antimicrobial-loaded phase-shift nanodroplet platform may be administered using any, or all, of the routes mentioned previously. The platform may then be allowed to distribute to the desired target area (i.e. site of biofilm infection) whereupon the target area is exposed to ultrasound at a frequency or intensity necessary to achieve the desired effect. Specifically, it is envisaged that the phase-shift nanodroplet platform will accumulate beyond the EPS matrix whereupon ultrasound exposure will simultaneously initiate dispersal of the EPS matrix whilst vaporizing the nanodroplet, thereby forming microbubbles. Subsequent ultrasound exposure at lower intensities can then rupture the microbubbles and release the antimicrobial which is then able to penetrate the target area (i.e. bacterial cells) to instigate therapeutic effect.

The therapeutically effective dose of the pharmaceutical composition herein described will depend on the nature of the platform, the antimicrobial utilised, the mode of administration, the condition to be treated, the patient, the target site etc. and may be adjusted accordingly by those skilled in the art.

The frequency and intensity of ultrasound necessary to achieve the desired effect will similarly depend on the nature of the platform, the condition to be treated, the patient, the target site etc. and may be based on the need to achieve selective dispersal of the biofilm and vaporization of the phase-shift nanodroplet at the target site. Ultrasound frequencies may vary depending on apparatus and will typically be in the range of 1 kHz to 10 MHz, either as a single frequency or a combination of different frequencies. Ultrasound intensities may range from 0.1 μW/cm² to 1 kW/cm². More than one ultrasound intensity may be used during a single treatment, i.e. a higher ultrasound intensity for biofilm dispersal and nanodroplet vaporization, and a lower ultrasound intensity for release of antimicrobials. Treatment time may range from 1 ms to 100 minutes and may be dependent on the intensity chosen (i.e. lower intensity will prolong treatment time). Ultrasound may be applied in continuous or pulsed mode or continuous mode and may be applied as either a focused or unfocused beam.

### EXAMPLES

This invention will now be described further with reference to the following non-limiting examples and accompanying drawings.

### Example 1—Preparation of Azithromycin-Loaded Phase-Shift Nanodroplets

To form azithromycin-loaded phase-shift nanodroplets, a protocol was adapted from patent information of the clinical microsphere agents, Definity™ and Definity RT™ (Lantheus Medical Imaging, N. Billerica, MA). Briefly, a 6.66×10⁻⁷ mol lipid mixture consisting of 81 mol % 1,2-distearoyl-sn-glycero-3-phosphocholine (DSPC) in a mixture of 90 vol % chloroform and 10 vol % methanol, 10 mol % 1,2-distearoyl-sn-glycero-3-phosphate (DSPA) in a mixture of 65 vol % chloroform, 35 vol % methanol, and 8 vol % MilliQ water, and 8 mol % 1,2-distearoyl-sn-glycero-3-phosphoethanolamine-N-[methoxy(polyethylene glycol)-5000 (DSPE-mPEG-5000) in a mixture of 90 vol % chloroform and 10 vol % methanol, were combined in 0.5 dram borosilicate glass vials (Ampulla, Hyde, UK). Azithromycin was dissolved in 90 vol % chloroform and 10 vol % dimethyl sulfoxide, added to the vial prior to the addition of the lipid mixture. Lipid solutions were slowly vortexed, dried under low-pressure nitrogen gas, and placed under vacuum to form a lipid film. Films were rehydrated with 1 mL 80:10:10 v/v acetate buffer (pH=5.2; 0.074 mg anhydrous sodium acetate and 0.006 mg glacial acetic acid in MilliQ), propylene glycol, and glycerol. Vials were heated at 60° C. using a water bath for 1 min, and sonicated (QSonica Q125, probe diameter 2 mm, 125 W, 20% intensity) using a probe sonicator for 3 minutes (30 s cycles ×6) to suspend the lipid film. The headspace within the vial was replaced with a 1:1 mixture of perfluoropropane (C₃F₈; PFP) and perfluorobutane gas (C₄F₁₀; PFB) for 15 seconds before mechanical agitation for 45s using a VialMix® shaker (Lantheus Medical Imaging, N. Billerica, MA) or similar. Samples were cooled for 10 minutes before use and were measured immediately to assess precursor microbubble size and concentration whilst mitigating temporal effects. Vials were then cooled in an ethanol salt ice bath (or 40 vol % ethylene glycol in water) at −10° C. under nitrogen pressure until the solution became clear, representing the condensation of the precursor microbubble to a nanoformulation state.

Following preparation as described above, Size and concentration of nanodroplets were determined using simultaneous interferometry (Videodrop; Meritics, Bedfordshire, UK), dynamic light scattering (DLS; Zetasizer Nano ZS; Malvern, Malvern, UK), and electro-impedance volumetric zone sensing (Coulter Counter Multisizer Z3; Beckman Coulter, Brea, CA). Coulter measurements were conducted using samples diluted in Isoton-I electrolyte solution (Beckman Coulter, Brea, CA), where a background count of buffer was taken and subtracted from the final count. Number and size distributions were measured using a 20-μm aperture, detecting diameters from 0.4-12 μm. Both interferometry and DLS measurements were conducted using samples diluted in deionized water. For the former, 5×60 second videos were collected and analyzed using the manufacturer's built-in software. Zeta potentials of nanodroplets were determined using dynamic light scattering (DLS; Zetasizer Nano ZS; Malvern, Malvern, UK), using samples diluted in 10 mM HEPES buffer (pH=7.4), 10 mM NaCl solution (pH=7.0), and 10 mM ammonium acetate solution (pH=7.0). Measurements were performed using the Smoluchowski protocol for up to 100 runs, with three measurements conducted per sample. Confocal and transmission electron microscopy (TEM) of nanodroplets was conducted to visualize ruthenium incorporation within the nanodroplet shell and the shape of formed nanodroplets, respectively. Confocal imaging was performed with a Zeiss 710 microscope using an oil-immersion objective lens.

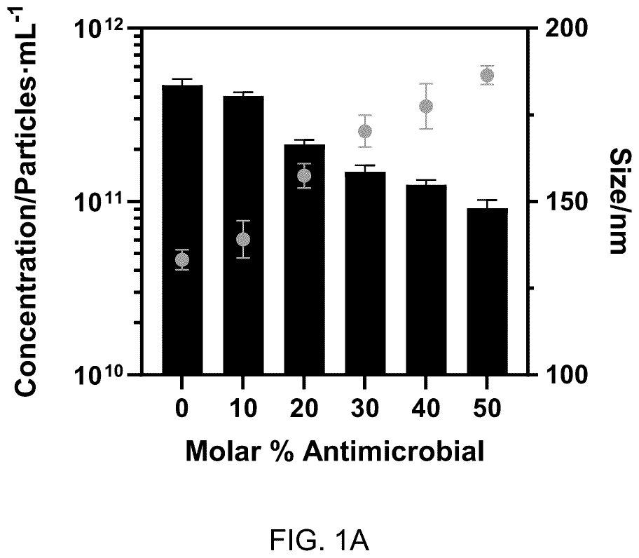

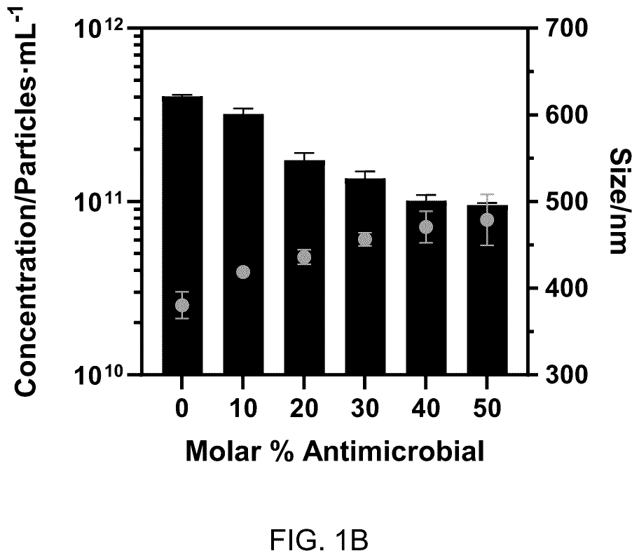

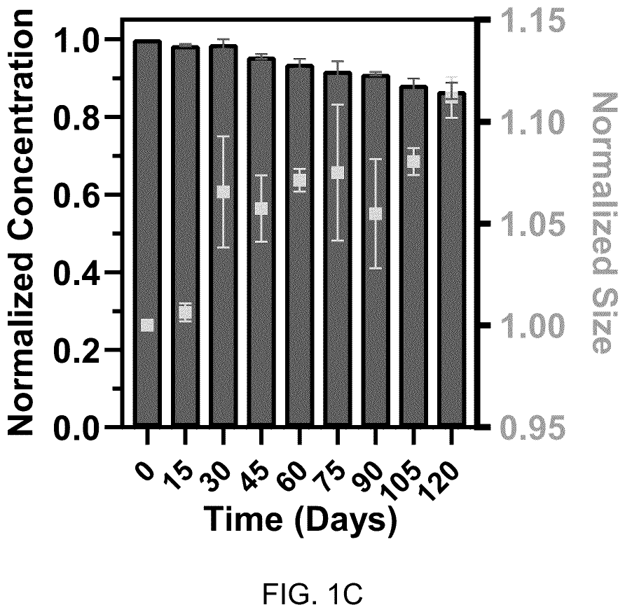

Azithromycin-loaded precursor microbubbles had an average diameter of 300-800 nm, average volume of 2-6 μm³, and concentration of approximately 10¹¹ particles/mL (FIG. 1B). When condensed, the resultant phase-shift nanodroplets had an average diameter of 100-300 nm, zeta potential of +10 to −30 mV, and concentration of approximately 10¹¹ particles/mL (FIG. 1A). Both precursor microbubbles and phase-shift nanodroplets could be observed to be formed with up to 50 mol % azithromycin within the overall formulation. To determine the effect that inclusion of azithromycin had on phase-shift nanodroplet stability in serum, azithromycin-loaded nanodroplets were diluted into either PBS or 50 vol % PBS/50 vol % fetal bovine serum (FBS) at 1×10⁹ nanodroplets/mL and kept at 37° C. to represent in vivo conditions. In evaluating stability of size and concentration, particle solutions were removed at 5, 15, 30, and 60 minutes, 2, 3, 4, 6, 8, and 24 hours for analysis using electro-impedance volumetric zone sensing, dynamic light scattering, and interferometry. In evaluating drug loading stability, 2.5 mL of diluted particle solution in 50 vol % FBS was loaded onto a 2K MWCO dialysis cassette (Thermo Scientific, Waltham, MA) as per manufacturer instructions. The cassette was floated vertically in a 100 mL beaker of deionized water, stirred at 100 rpm and heated to 37° C. 1 mL samples were taken from the release medium at 5, 15, 30, and 60 minutes, 2, 3, 4, 6, 8, and 24 hours, with the same volume of dialysis buffer added back to maintain sink conditions. Samples were digested with 65 vol % nitric acid for 22 hours, followed by 2 hours at 40° C. Digests were diluted to 2 vol % aqueous nitric acid with deionized water and quantified on ICP-MS (NexION 2000B ICP-MS, PerkinElmer). Sample solution was mixed with carrier (2% v/v nitric acid) and internal standard (In, 1 ng/g) before aspiration. The results indicate that 40 mol % azithromycin-loaded phase-shift nanodroplets retain up to 90% of their initial concentration after 100 hours incubation, with less than 1% of the population being above 0.8 μm, representing spontaneous vaporization. Similarly, less than 10% of the loaded azithromycin was observed to leak prematurely within 48 hours when incubated. Prepared pharmaceutical compositions were also observed to be stable for 120 days when stored at room temperature (FIG. 1C).

### Example 2—Preparation of Besifloxacin-Loaded Phase-Shift Nanodroplets

To form besifloxacin-loaded phase-shift nanodroplets, a similar protocol as previously mentioned in example 1 was utilized. Specifically, the loading of besifloxacin onto the phase-shift nanodroplet was accomplished by dissolving besifloxacin hydrochloride in a 10 vol % benzyl alcohol, 10% dimethyl sulfoxide and 80% chloroform solution and adding the agent into the vial prior to the addition of the lipid mixture. All other steps as mentioned previously remained the same.

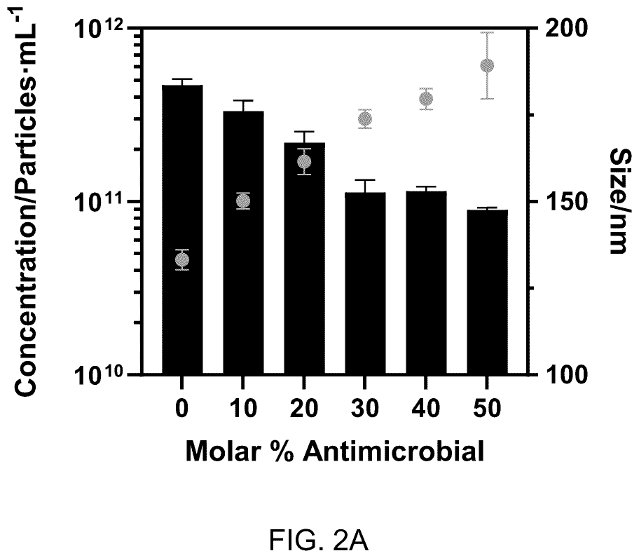

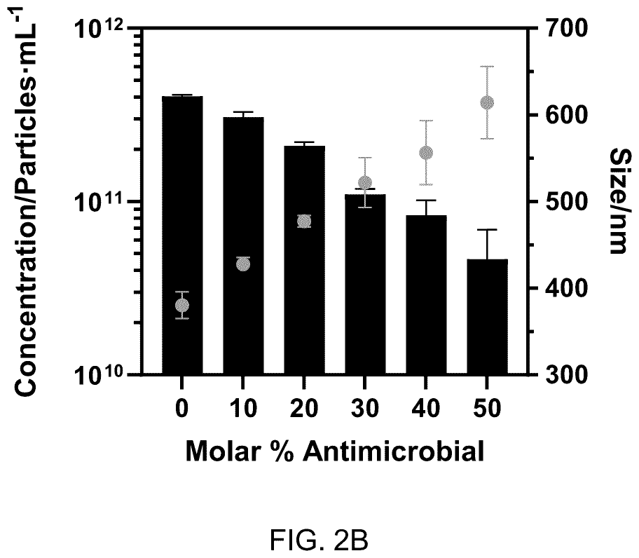

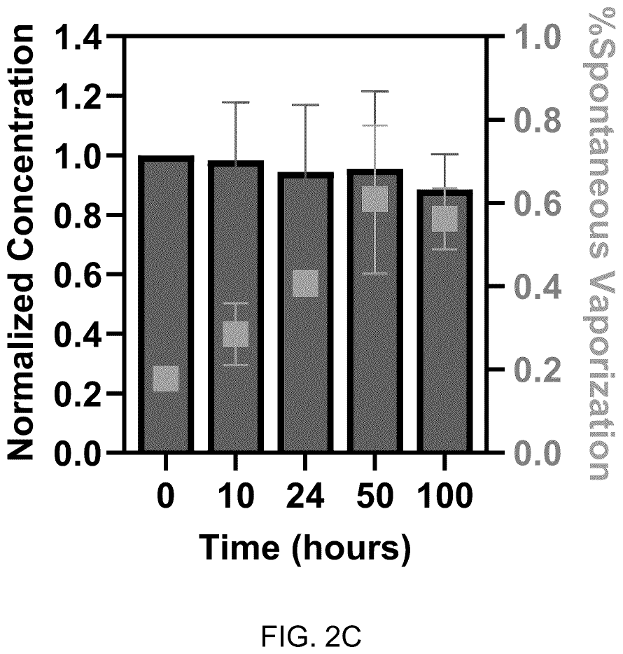

Besifloxacin-loaded precursor microbubbles had an average diameter of 400-1500 μm, average volume of 2-8 μm³, and concentration of approximately 10¹¹ particles/mL (FIG. 2B). When condensed, the resultant phase-shift nanodroplets had an average diameter of 100-300 nm, zeta potential of +10 to −30 mV, and concentration of approximately 10¹¹ particles/mL (FIG. 2A). Both precursor microbubbles and phase-shift nanodroplets could be observed to be formed with up to 50 mol % besifloxacin within the overall formulation. When evaluating the stability of besifloxacin-loaded phase-shift nanodroplets in serum, results indicate that up to 80% of the initial concentration was retained after 100 hours incubation, with less than 1% of the population being above 0.8 μm, representing spontaneous vaporization (FIG. 2C). Less than 15% of the loaded besifloxacin was observed to leak prematurely within 48 hours when incubated. Prepared pharmaceutical compositions were also observed to be stable for 120 days when stored at room temperature.

### Example 3—Preparation of Polymyxin B-Loaded Phase-Shift Nanodroplets

To form polymyxin B-loaded phase-shift nanodroplets, a similar protocol as previously mentioned in example 1 was utilized. Specifically, the loading of polymyxin B onto the phase-shift nanodroplet platform was accomplished by dissolving polymyxin B in a 10 vol % dimethyl sulfoxide and 90 vol % chloroform solution and adding the agent into the vial prior to the addition of the lipid mixture. All other steps as mentioned previously remained the same.

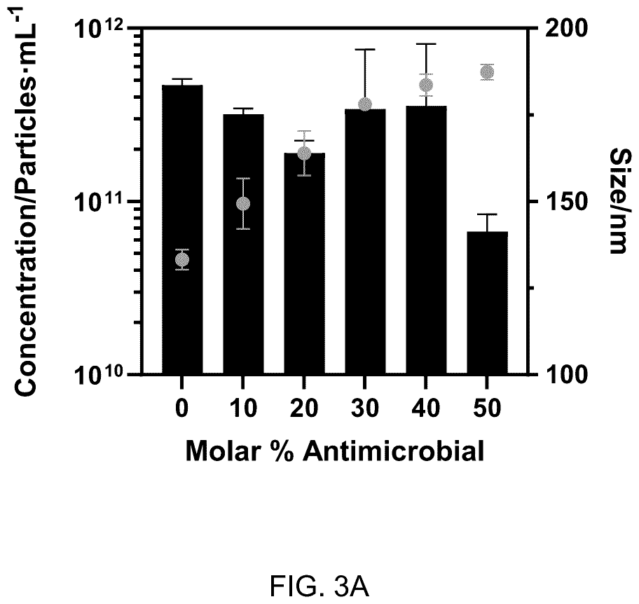

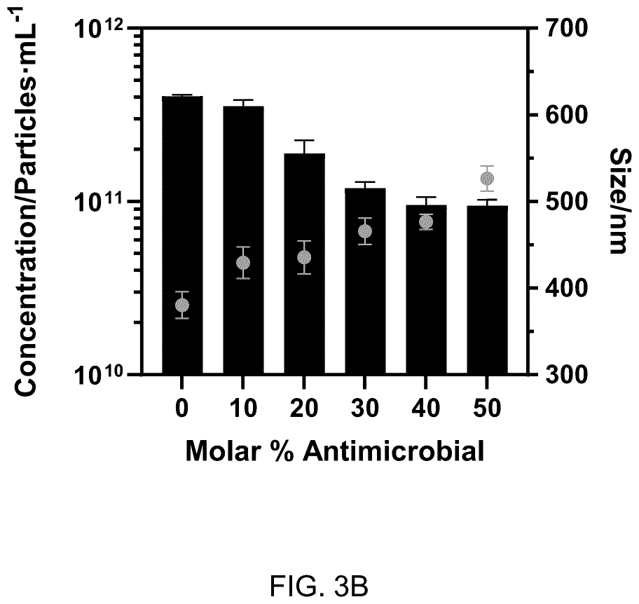

Polymyxin B-loaded precursor microbubbles had an average diameter of 400-800 μm, average volume of 2-6 μm³, and concentration of approximately 10¹¹ particles/mL (FIG. 3B). When condensed, the resultant phase-shift nanodroplets had an average diameter of 100-300 nm, zeta potential of +10 to −20 mV, and concentration of approximately 10¹¹ particles/mL (FIG. 3A). Both precursor microbubbles and phase-shift nanodroplets could be observed to be formed with up to 50 mol % polymyxin B within the overall formulation. When evaluating the stability of polymyxin B-loaded phase-shift nanodroplets in serum, results indicate that up to 80% of the initial concentration was retained after 50 hours incubation, with less than 1% of the population being above 0.8 μm, representing spontaneous vaporization. Less than 15% of the loaded besifloxacin was observed to leak prematurely within 48 hours when incubated. Prepared pharmaceutical compositions were also observed to be stable for 120 days when stored at room temperature.

### Example 4—Preparation of Ruthenium Polypyridyl-Loaded Phase-Shift Nanodroplets

To form ruthenium polypyridyl-loaded phase-shift nanodroplets, a similar protocol as previously mentioned in example 1 was utilized. Specifically, the loading of ruthenium polypyridyl complexes onto the phase-shift nanodroplet platform was accomplished by reacting ruthenium polypyridyl complex to 1,2-distearoyl-sn-glycero-3-phosphoethanolamine-n-[dibenzocycooctyl(polyethylene glycol)-5000](DSPE-PEG (5000) DBCO) in 10 vol % acetonitrile and 90 vol % chloroform overnight at 37° C. under 200 rpm mixing. The resultant lipid complex was then dissolved in a 5 vol % acetonitrile and 95 vol % chloroform solution and added into the vial prior to the addition of the lipid mixture. All other steps as mentioned previously remained the same.

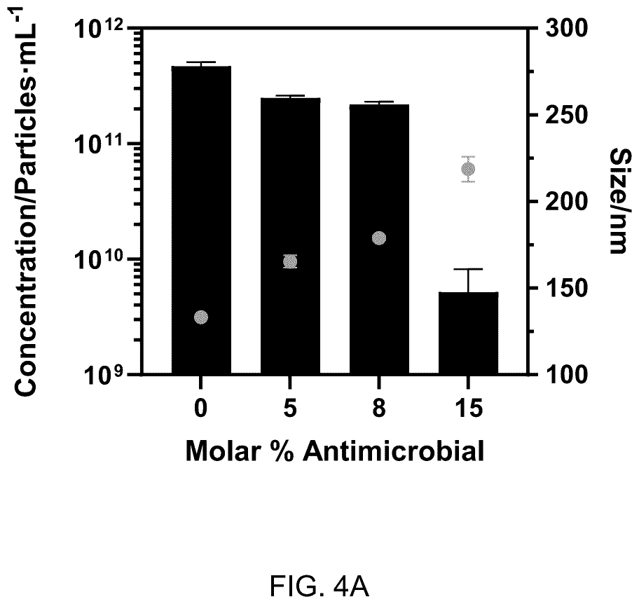

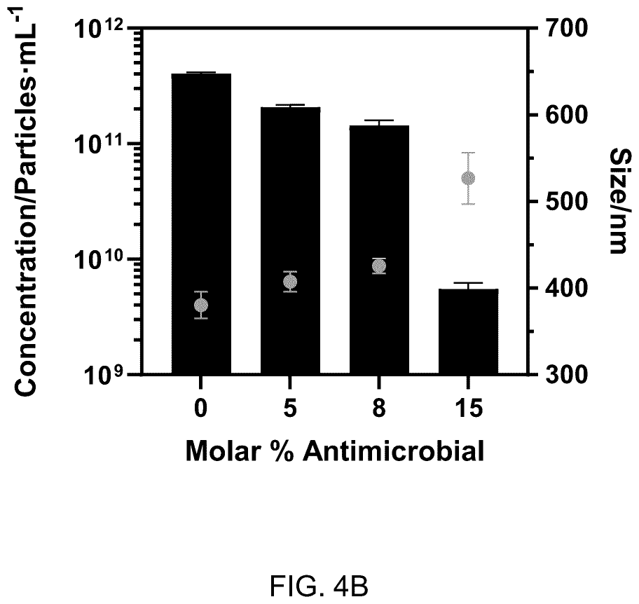

Ruthenium polypyridyl complex-loaded precursor microbubbles had an average diameter of 500-900 μm, average volume of 2-7 μm³, and concentration of approximately 10¹¹ particles/mL (FIG. 4B). When condensed, the resultant phase-shift nanodroplets had an average diameter of 200-400 nm, zeta potential of +10 to −30 mV, and concentration of approximately 10¹¹ particles/mL (FIG. 4A). Both precursor microbubbles and phase-shift nanodroplets could be observed to be formed with up to 8 mol % ruthenium polypyridyl complex within the overall formulation. When evaluating the stability of ruthenium polypyridyl complex-loaded phase-shift nanodroplets in serum, results indicate that up to 85% of the initial concentration was retained after 100 hours incubation, with less than 1% of the population being above 0.8 μm, representing spontaneous vaporization. Less than 15% of the loaded ruthenium polypyridyl complex was observed to leak prematurely within 48 hours when incubated. Prepared pharmaceutical compositions were also observed to be stable for 120 days when stored at room temperature.

### Example 5—Acoustic Characterization of Antimicrobial-Loaded Phase-Shift Nanodroplets

To determine acoustic vaporization thresholds, high-speed imaging was used in-line with methods previously reported. Briefly, vaporization was defined as the appearance in gray-scale contrast in the optical focal region above that of noise, as defined by pixel counts during exposure compared with before ultrasound exposure. As acoustic pressures were increased stepwise, any statistically significant increase in contrast compared to the previous pressure was defined as an increase in acoustic vaporization. For each acoustic pressure, nanodroplets were flowed through using a syringe pump at a constant rate of 0.36 mL/min.

The results shown indicate that a vaporization threshold could be observed following ultrasound exposure when driven at its center frequency, 3.125 MHz, with a frequency bandwidth of ⅓, at a focal point of [−3.8 mm, 0.2 mm, 190 mm]. The arbitrary transmit waveform comprised of a 99.0% active transmit driver during the half cycle period, a 20-cycle burst, and positive polarity during the first half-cycle with equalization pulses added to the start and the end of the burst. Specifically, the vaporization threshold was found to be between 2.5 to 3.5 MPa for all tested formulations.

### Example 6—Planktonic Cytotoxicity Experiments

The antimicrobial inhibitory properties of the nanodroplet platform were assayed using the broth microdilution reference method per ISO 20776-1 recommendations. Briefly, S. aureus and E. coli isolates were streaked, cultured, and diluted in un-supplemented Mueller-Hinton broth to a final inoculum of 5×10⁵ CFU/mL, as determined by optical density calibrations. Bacterial suspensions were treated at different antimicrobial molar concentrations either with free drug, drug-loaded nanodroplets alone, or drug-loaded nanodroplets exposed to ultrasound. Microplates were incubated at 37° C. for 20 hours before visual confirmation of minimum inhibitory concentrations (MIC). As defined by EUCAST, MIC was read as the lowest concentration of antimicrobial agent that completely inhibited the growth of the organism, by way of turbidity, as detected by the unaided eye. Metabolic inhibitory measurements were conducted following growth in either brain heart infusion (S. aureus) or synthetic human urine (E. coli) using 5% v/v alamarBlue™ (resazurin). As prior, initial inoculi of 5×10⁵ CFU/mL, as determined by optical density calibrations, were treated with equivalent drug molar concentrations and diluted serially with PBS prior to incubation at 37° C. for 20 hours. Resazurin was then added and incubated for 1 hour, and measured on fluorescence (excitation 540 nm, emission 560-620 nm). Wells on each row with culture medium without cells were used as negative sterility controls, and wells with inoculum and no antimicrobial were used as growth controls. Each drug-isolate combination concentration gradient was repeated in duplicate. The calculation of the concentration required to inhibit the net increase in metabolic viability by 50% was calculated from a dose response curve by nonlinear regression, and are expressed as mean values ±SEM. As all dose response curves produced a biphasic or triphasic response due to the presence of dormant persister cells, only the first curve, corresponding to metabolically active cells, was taken into consideration for calculations. Bactericidal efficacy was assessed using agar plate microdilution subculture of stationary phase (OD=0.8) S. aureus (4.74×10⁸ CFU/mL) and E. coli (3.84×10⁸ CFU/mL) in brain heart infusion and synthetic human urine, respectively. Bacterial cultures were treated at different antimicrobial molar concentrations either with free drug or drug-loaded nanodroplets for a 24-hour incubation period before ultrasound exposure, if applicable, was performed using previously mentioned parameters. Following ultrasound exposure, bacterial suspensions were incubated for a further 3 hours to allow any relevant antimicrobial mechanisms of action to occur. Bacterial suspensions were then centrifuged at 5000 RCF for 10 min to remove the remaining drug before resuspending in PBS and minimize antibiotic carryover. Samples from wells were serially diluted and individually plated on 1.5% w/v agar Luria-Bertani (LB) under standardized conditions as described in document M26-A. The minimum bactericidal concentration (MBC) was defined as the concentration required to kill 99.9% of counted colonies relative to the untreated negative growth control following a 24-hour incubation period, as calculated from a dose response curve by nonlinear regression. Each sub-culture was plated in triplicate as a technical replicate and each drug-isolate combination concentration gradient was repeated in duplicate as a biological replicate. As before, dose response curves exhibited a multiphasic response, and in some cases a paradoxical effect phenomenon, necessitating that only the first curve, corresponding to metabolically active cells, was taken into consideration for calculations.

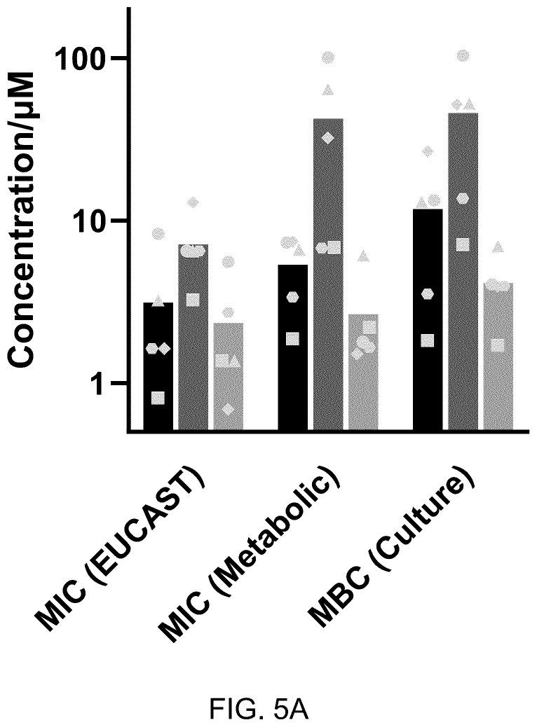

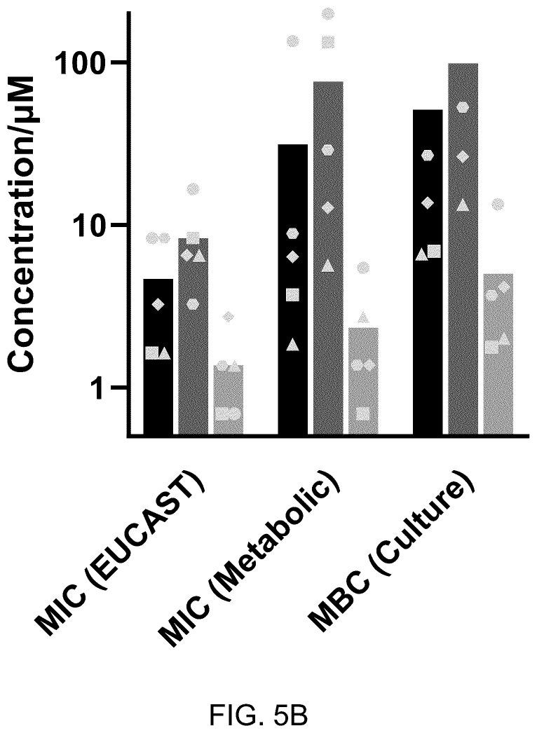

The results reveal that a statistically significant reduction in viability was observed across all tested clinical isolates for both S. aureus (FIG. 5A) and E. coli (FIG. 5B) treated with combined antimicrobial-loaded phase-shift nanodroplets and focused ultrasound, when compared with either antimicrobial alone, antimicrobial-loaded nanodroplets alone, or ultrasound alone. Furthermore, either no statistically significant decrease in cell viability, or an increase in cell viability, could be observed when comparing antimicrobial-loaded nanodroplets alone to antimicrobial alone. This trend could be observed across a range of antimicrobial concentrations.

### Example 7—Planktonic Cellular Uptake

S. aureus and E. coli isolates were streaked, cultured, and diluted in either brain heart infusion or synthetic human urine, respectively, in stationary phase to a final inoculum of 1×10⁸ CFU/mL. Free drug or antimicrobial-loaded nanodroplets at 0.50 μM were pipetted into each inoculum suspension and incubated at 37° C. for 24 hours. Cell suspensions were then washed by centrifugation at 5000 RCF, 4° C. for 10 minutes. Cell pellets were resuspended in 1 mL PBS before 50 μL was removed for analysis as the whole cell fraction. The remaining fraction was pelleted once more before being resuspended in buffer solution. For E. coli, cells were first incubated in a concentrated EDTA-sucrose solution (4° C. 950 μL buffer with 100 mM Tris-acetate, 500 mM sucrose, 5 mM ethylenediaminetetraacetic acid, 1 mM MgCl₂ in deionized water) to destabilize the outer membrane before adding 50 μL 2 mg/mL lysozyme in TE buffer to cleave the periplasmic peptidoglycan layer. This was incubated for 5 minutes at 37° C. before a final mild osmotic shock (20 μL 1M MgSO₄) was performed to finalize peptidoglycan hydrolysis. For S. aureus, cells were instead incubated in a Tris Sucrose-Magnesium chloride (TSM) buffer (10 mM MgCl2, 500 mM Sucrose in 50 mM Tris, pH 7.5) with 0.2 μg/mL lysostaphin and 1× Protease inhibitor for 30 minutes at 37° C. to isolate the cell wall fraction. In both cases, the suspension was then centrifuged at 3200 RCF for 10 minutes at 4° C., collecting the supernatant as either the periplasmic or cell wall fraction for analysis. The remaining cell pellet was re-suspended in 4° C. 1 mL lysis buffer, 2 μl/mL DNase, and 2 μL 0.5 M EDTA, incubating under agitation at 300 rpm for 15 minutes. Cell suspensions then underwent three cycles of freeze-thaw, going between −70° C. and 37° C., with a minimum of 1 hour at each temperature. To confirm successful cell lysis through this procedure, brightfield microscopy images were taken on select samples. Samples were then pelleted at 16000 RCF for 72 hours, to maintain equivalence with higher G-force rates at lower times, at 4° C. before removing the supernatant (cytoplasmic/protoplast fraction) and pellet (membrane bound fraction) for analysis. All samples were concentrated using a SpeedVac and digested in 65% w/v nitric acid for a minimum of 24 h, of which 2 h were spent under 60° C. heat. Solution samples were then diluted in MilliQ water to a final nitric acid concentration of 5% w/v, such that observed values were within the range of sensitivity of the machine. Diluted samples were passed through a 0.45 μm polytetrafluoroethylene (PTFE) membrane filter. A calibration curve for both elemental europium and ruthenium generated from certified reference materials was used to quantify the concentration of metal in each sample. To back-calculate for administered dose, a further 10 μL sample of the administered agent was also digested and diluted under similar protocols. All reported uptake concentrations were calculated based on the measured administered dose as assessed through a NexION 5000 ICP-MS (PerkinElmer) with autosampler. Sample solution was mixed with carrier (5% v/v HNO3) and internal standard (In, 1 ng/g) before aspiration. Quality control standards were performed every 20 samples and monitored for deviation.

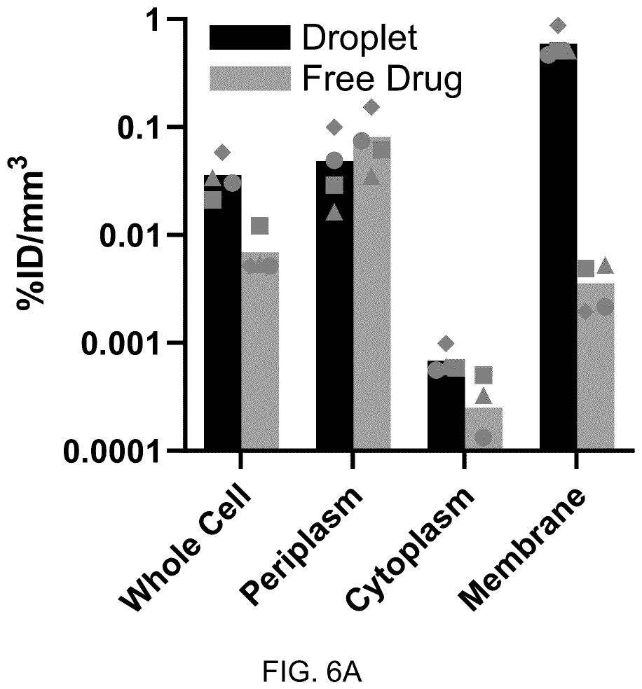

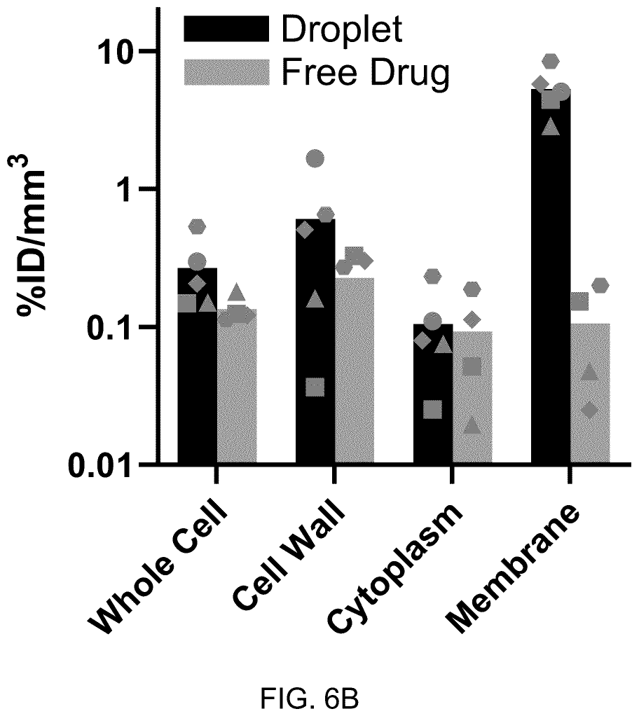

Results indicate that a statistically significant increase in overall antimicrobial cellular uptake could be observed when planktonic bacteria were incubated with antimicrobial-loaded phase-shift nanodroplets when compared to either antimicrobial alone in E. coli (FIG. 6A) or S. aureus (FIG. 6B). Similar results were also observed when isolating for cytoplasmic uptake, representing the location whereupon many antimicrobials exert their mechanisms of action.

### Example 8—Biofilm Cytotoxicity

Biofilms were grown using an interlaboratory validated modified Calgary Biofilm Device (ThermoFisher; Waltham, MA) or a similar custom-peg lid design in the case of ultrasound-exposed biofilms. Both allowed for biofilm growth on removable polypropylene pegs with a high surface area to volume ratio with similar growth kinetics. Both S. aureus and E. coli biofilms were grown using a starting inoculum of 6.1×10⁶ CFU/mL and 7.96×10⁶ CFU/mL (OD=0.01) in brain heart infusion and synthetic human urine, respectively, before incubating at 37° C. for 72 hours, with media changes every 24 hours. Biofilms were treated at different antimicrobial molar concentrations either with free drug or drug-loaded nanodroplets for a 24-hour incubation period before ultrasound exposure, if applicable, was performed using previously mentioned parameters. Following ultrasound exposure, bacterial suspensions were incubated for a further 3 hours to allow any relevant antimicrobial mechanisms of action to occur. For biomass experiments, pegs were air dried for 30 minutes before being transferred to a 0.1% safranin solution and stained for 10 minutes. Pegs were subsequently removed and washed twice by submerging in deionized water to remove unbound stain before once again air drying for a further 30 minutes. Safranin was removed by solubilizing pegs in 33% acetic acid for 30 minutes and measured on absorbance (520 nm). For metabolic viability and culturability assays, pegs were air dried for 30 minutes before being solubilized in 4° C. 5 mM ethylenediaminetetraacetic acid in deionized water for 30 minutes to stimulate nutrient deprivation and biofilm dispersal. Biofilms were then further dislodged by scraping before adding 5% v/v alamarBlue™ (resazurin). Stained bacterial suspensions were incubated for 2.5 hours before measuring on fluorescence in triplicate (excitation 540 nm, emission 560-620 nm). Each aliquot was then plated on 1.5% w/v agar Luria-Bertani (LB) and incubated for 24 hours. As before, the minimum biofilm eradication concentration (MBEC) was defined as the concentration required to kill 99.9% of counted colonies or reduce resazurin signal by 80% relative to the untreated negative growth control following a 24-hour incubation period, as calculated from a dose response curve by nonlinear regression. Each drug-isolate combination concentration gradient was repeated in triplicate as a biological replicate.

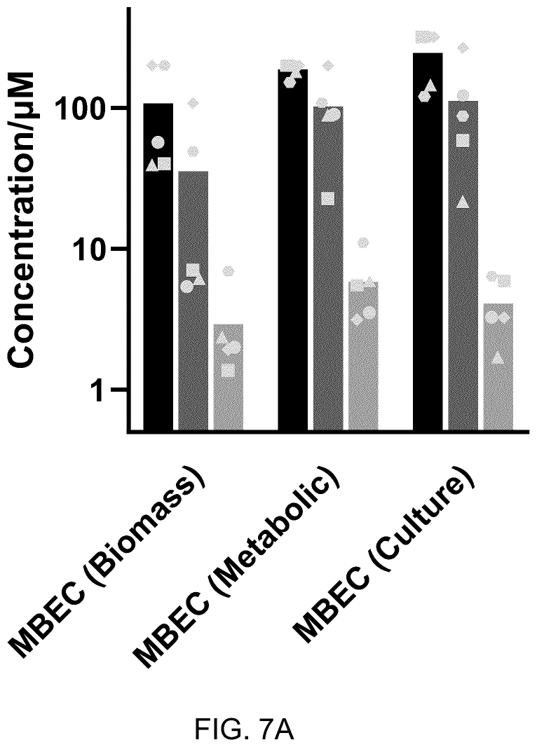

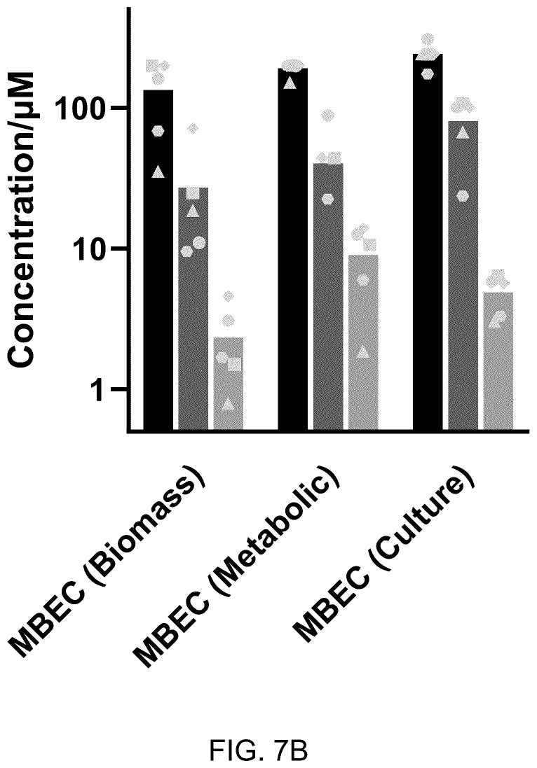

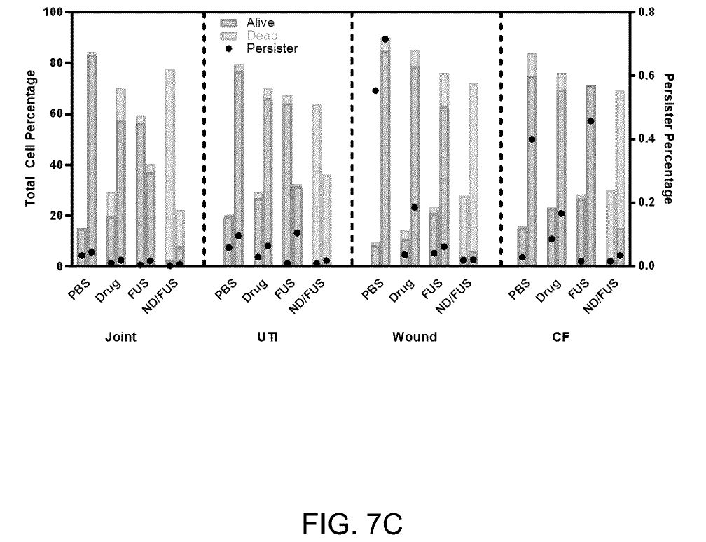

Biofilm eradication measurements with response to focused ultrasound was conducted using a modified Ibidi μ-slide I Luer flow cell system, connected to media and waste carboys by silicone rubber tubing, and either statically grown or flowed through using a syringe pump. The flow cell was inoculated with 6.1×10⁵ CFU/mL (Calibrated McFarland Standard=OD 0.5) of either S. aureus or E. coli at the logarithmic stage of growth and allowed to attach to the polymer surface for 30 minutes before flow was started. Thereafter, influent flow was begun at 0.6 mL/hr and was incubated at 37° C. for 72 hours. Confirmation of successful biofilm formation was done under brightfield microscopy and verification of this procedure was performed on select samples using safranin and concanavalin A lectin staining. At the end of the 72-hour growth period, the flow cell system was placed within a custom-built sample holder inside an ultrasound setup. Free drug, lipid suspension, or nanodroplets were diluted with PBS to an equivalent concentration of 1×10⁹ nanodroplets/mL and flowed into the system at 0.2 mL/min for 5 minutes. In experiments involving ultrasound, samples were exposed using the treatment scheme defined previously. Following treatment, flow cells were immediately flushed back and forth repeatedly with 0.5 M EDTA to dissociate any remnant cells attached to the polymer surface. Removal of the cells and EPS matrix was confirmed on confocal microscopy on select samples by concanavalin A lectin and Syto9 staining. Cell suspensions were then washed three times through centrifugation for 10 minutes at 4000 rpm with PBS before vortexing and replacing with fresh media. Cells were then left in the incubator for 24 hours to revive any dormant cells. To assess metabolic viability, the BacLight™ RedoxSensor™ Green reagent (ThermoFisher, Waltham, MA) and PI were used per manufacturer instructions, using sodium azide and carbonyl cyanide chlorophenylhydrazone (CCCP) as positive control reagents for Gram-negative and Grampositive strains, respectively. After staining, both initial outflow and remaining biofilm samples were fixed using 4% paraformaldehyde (Biolegend, San Diego, CA) per manufacturer instructions, and analysed on the BD LSRFortessa™ flow cytometer for 60 seconds at “low” (12±3 μL/min). Bacterial populations/millilitre were defined as the number of events recorded within the FSC/SSC preset gate and from the volume aspirated by the flow cytometer over the run time. Bacterial populations were further verified by assessing the preset PI gate, set to evaluate membrane permeable dead cells. Ultrasound-mediated membrane permeability was mitigated by waiting 4 hours post-treatment to allow for transient poration or other structural perturbation to recover. Metabolically active cells were defined as the number of events recorded within a “positive” RedoxSensor Green gate from the bacterial populations identified. Dead and persister cells were defined as the number of events recorded within a “negative” RedoxSensor Green gate from the bacterial populations identified. This was further segregated by defining dead cells to be the number of events recorded with both a “very negative” RedoxSensor Green gate and a “strong positive” PI gate; likewise, persister cells were defined as the number of events recorded with a “strong negative” RedoxSensor Green gate and a “strong negative” DAPI gate. Controls for persister populations were generated through 100× MIC ampicillin. To confirm results outputted through FACS, culturability assays were further performed on both the initial outflow and remaining biofilm samples by plating an aliquot of cell suspension after washing on 1.5% w/v agar Luria-Bertani (LB) and incubating for 24 hours. The results reveal that a statistically significant reduction in viability was observed across all tested clinical isolates for both S. aureus (FIG. 7A) and E. coli (FIG. 7B) treated with combined antimicrobial-loaded phase-shift nanodroplets and focused ultrasound, when compared with antimicrobial alone (FIG. 7C).

### Example 9—Biofilm Cellular Uptake

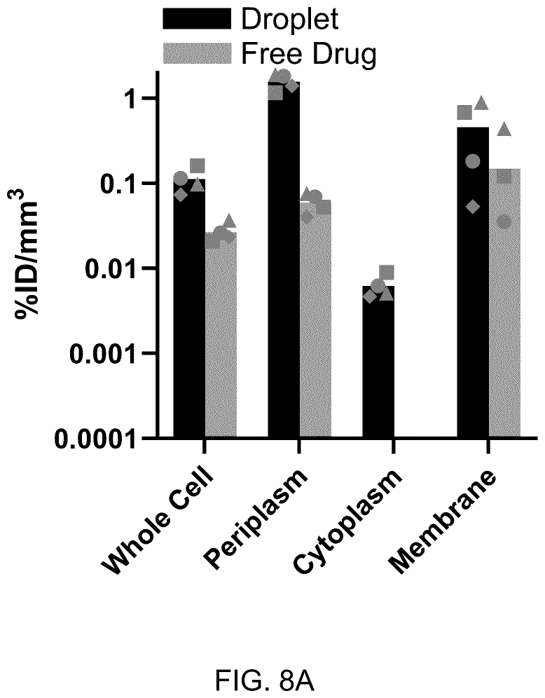

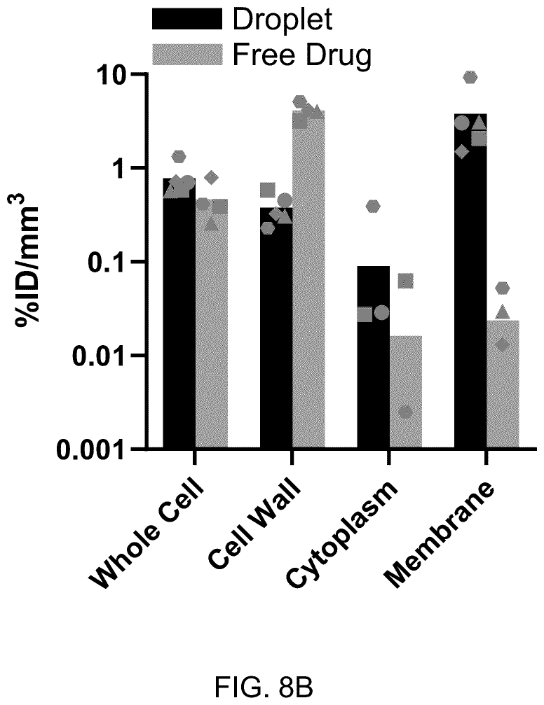

Cellular uptake measurements were conducted using similarly grown S. aureus and E. coli clinical isolate biofilms in Calgary Biofilm Devices as per previous. Peg-lids were treated using free drug, lipid suspension, or nanodroplets at subtherapeutic concentrations, where cytotoxicity was below 2% across all groups for 10 minutes or 24 hours. Peg-lids were washed by submerging in a microplate of phosphate-buffered saline before biofilms were dispersed using either 0.5 M EDTA or high-powered sonication. Resultant bacterial suspensions were then collected and centrifuged at 5000×g for 10 minutes at 4° C., separating the supernatant and the pellet. The pellet was separated into subcellular components as detailed previously whereas the supernatant was centrifuged further at 12000× g for 30 minutes at 4° C. to remove residual entrained cells before precipitating by adding cold ethanol in a 3:1 ratio and storing at −20° C. overnight. The precipitate was then pelleted by centrifuging at 12000× g for 30 minutes at 4° C. before collecting the pellet as the exopolysaccharide polymers. Results indicate a significantly higher accumulation within the cytoplasmic compartment of biofilm-residing bacteria when comparing nanodroplet-encapsulated antimicrobials to free drug formulations in E. coli (FIG. 8A) and S. aureus (FIG. 8B).

### Example 10—Persister Cell Cytotoxicity

S. aureus and E. coli clinical isolate suspensions were prepared as previously stated before being treated with either free drug or antimicrobial-loaded nanodroplets, with or without ultrasound. Treated cultures were then isolated for persister cells by rapidly killing normally growing cells using 200 μL cell lysis solution (Sigma Aldrich, Gillingham) and incubating for 10 minutes. The suspension was further isolated by adding 200 μL of enzymatic lysis solution (50 mg/mL lysozyme in TE buffer) and incubating for 15 minutes. The lytic mixture was washed out and concentrated with phosphate-buffered saline and replaced by fresh media before being serially diluted and plated on LB plates for determination of persister cell frequencies. Results show a significant reduction in required antimicrobial concentrations to achieve complete cell eradication when compared to free antibiotic.

### Example 11—Preparation of Minocycline-Loaded Nanodroplet Gels

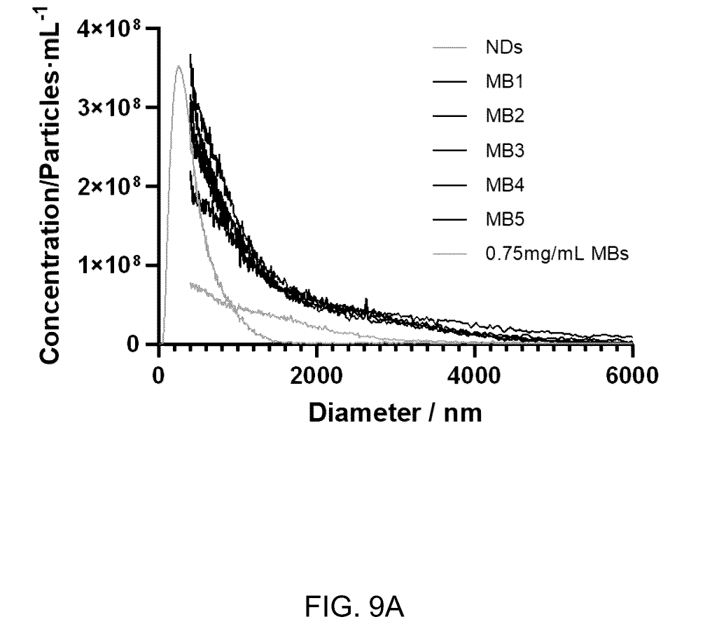

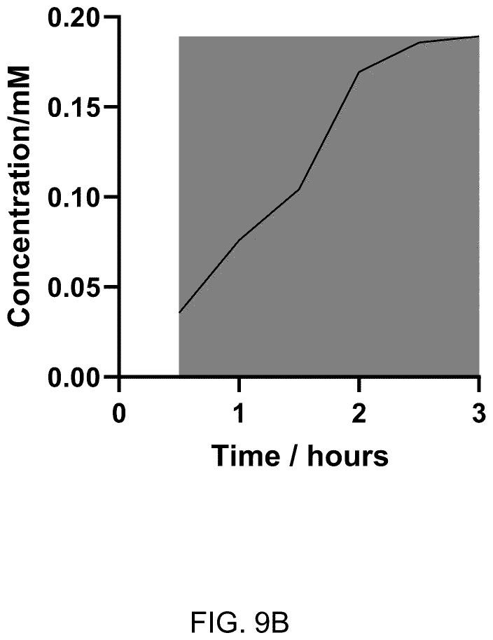

To form minocycline-loaded phase-shift nanodroplets, a similar protocol as previously mentioned in example 1 was utilized. Specifically, the loading of minocycline onto the phase-shift nanodroplet platform was accomplished by dissolving minocycline in a methanolic solution and adding the agent into the vial prior to the addition of the lipid mixture. Lipid concentrations were maintained at 4.32×10⁻⁶ mol. All other steps as mentioned previously remained the same. Minocycline-loaded precursor microbubbles had an average diameter of 400-800 μm, average volume of 2-6 μm³, and concentration of approximately 10¹¹ particles/mL. When condensed, the resultant phase-shift nanodroplets had an average diameter of 100-300 nm, zeta potential of +10 to −30 mV, and concentration of approximately 10¹² particles/mL (FIG. 9A).

Physically crosslinked nanodroplet gels were formulated by dissolving 2% w/w 700-1000 kDa hyaluronic acid, 2% w/w panthenol, and 0.4% w/w allantoin within the nanodroplet solution over the course of 3 hours at 4° C. Gels had a pH of 5-7 and exhibited shear thinning behavior in standard rheology testing. Franz diffusion cell testing through a model biofilm suggests passive release of nanodroplets from the gel through the biofilm within 2 hours of incubation (FIG. 9B).

### General Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the terms “patient” or “subject” are used interchangeably. The patient or subject treated by the presently disclosed compositions and methods is desirably a human subject, although it is to be understood that the methods described herein are effective with respect to all vertebrate species, which are intended to be included in the term “subject.” Accordingly, a “subject” can include a human subject for medical purposes (e.g., the diagnosis or treatment of an existing disease, disorder, or condition, or the prophylactic diagnosis or treatment for preventing the onset of a disease, disorder, or condition).

### REFERENCE STATEMENT

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

What is claimed:

1. A phase-shift nanodroplet-antimicrobial agent platform effective for treating a biofilm-related disease in a subject in need thereof.

2. The platform of claim 1, wherein the phase-shift nanodroplet is attached to or associated with at least one antimicrobial agent by covalent or non-covalent means.

3. The platform of claim 2, wherein the phase-shift nanodroplet comprises a shell and a liquid core, and wherein the shell comprises a material capable of forming and maintaining a layer at an interface between the liquid core and an external medium.

4. The platform of claim 3, wherein the material is a surfactant or a polymer.

5. The platform of claim 1, having a diameter in the range of 1 nm to 10 μm.

6. The platform of claim 1, wherein the biofilm-related disease is caused by a microbe such as a bacterium, a virus, a fungus, or a parasite.

7. The platform of claim 6, wherein the biofilm-related disease comprises pneumonia, cystic fibrosis, otitis media, chronic obstructive pulmonary disease, osteomyelitis, acne vulgaris, a urinary tract infection, a periodontal disease, and/or a medical device-related infection.

8. A pharmaceutical composition comprising the antimicrobial-loaded phase-shift nanodroplet platform of claim 1, and at least one pharmaceutical carrier or excipient, wherein the pharmaceutical composition is formulated for parenteral, oral, intranasal, intraarticular, or topical administration.

9. A method for treating a biofilm-related disease in a subject in need thereof, comprising:
a. administering an effective amount of the pharmaceutical composition of claim 8 to a treatment area in or on the subject; and
b. subjecting the treatment area to ultrasound irradiation.
